(19) United States
(12) Patent Application Publication
(10) Pub. No.: US 2005/0113762 A1
(43) Pub. Date: May 26, 2005

(54) Minimally invasive high viscosity material delivery system

(76) Inventors: John F. Kay, Encinitas, CA (US); Michael F. Sickler, Laguna Hills, CA (US)

Correspondence Address: John P. Iwanicki, Esq.;Banner & Witcoff, Ltd., 28th Floor, 28 State Street, Boston, MA, 02109, US

(21) Appl. No.: 10/721,637

(22) Filed: Nov. 24, 2003

(51) Int. Cl. A61F 2/00

(52) U.S. Cl. 604/181; 606/92; 606/93; 606/94

(57) ABSTRACT

A minimally invasive high viscosity material delivery system having a cannula associated with a dispenser for dispensing a high viscosity material out of the cannula, a body member having a first opening that is in communication with the cannula, a reservoir for receiving the high viscosity material, and a second opening associated with a transfer member for pushing the high viscosity material from the body member into the cannula via the first opening. The transfer member is in communication with the second opening. The cannula and the body member are connected in a non-linear angle. Also disclosed is a method of using such system.

### FIELD

Described herein are delivery devices suitable for introduction of high viscosity materials into the body. More specifically, the described delivery systems are particularly suitable for the delivery of high viscosity materials into constricted areas, and as such, are particularly useful in surgical procedures.

### BACKGROUND INFORMATION

Tissue regeneration materials may be used to fill bone defects to effect bone grafts. For better and faster recovery, it may be desirable to minimize the size of the surgical incisions required for the delivery of those tissue regeneration materials to the desired site. Since the tissue regeneration materials often have high viscosity, they are difficult to deliver to the surgical site using conventional delivery devices such as syringes. High viscosity materials are difficult to force out of a conventional syringe fitted with a needle. As a practical matter, conventional syringes when used alone (i.e., without a needle) are usually too large or too short for insertion into small surgical incisions. Smaller (or “down-sized”) syringes are often difficult to control when applying the high forces necessary to press viscous materials through the small exit bore. Furthermore, conventional syringes, whether used with or without needles, may be inefficient in that they often retain a portion of the tissue regeneration material inside the syringe body or needle.

The device described herein is able to deliver a high viscosity material to a constricted area with good control and to reduce the amount of wasted or undeliverable material that would otherwise remain within the delivery device.

### SUMMARY

Described here is a minimally invasive, high viscosity material delivery system suitable for delivering a high viscosity material to a constricted area. An example of such a use or procedure is the delivery of a tissue regeneration material through a small surgical incision and into the graft site, or the like.

When used properly, the system can reduce the amount of material that would otherwise remain within the delivery system.

The delivery system, due to its design, is easy to manipulate and to control when dispensing high viscosity material. The system design may also be configured to be low cost and perhaps disposable. This is an advantage when prevention of cross-contamination or a desire for avoiding “clean up” are significant design parameters.

In the most general terms, the described device includes a minimally invasive, high viscosity material delivery system comprising: a.) a cannula associated with a pressure applicator for dispensing a high viscosity material from the cannula, b.) a body member having (i) a first opening that is in fluid communication with the cannula, (ii) a reservoir for receiving the high viscosity material, (iii) a second opening allowing transfer of the high viscosity material into the reservoir, and (iv) a transfer member typically situated in the second opening that transfers or pushes high viscosity material from the reservoir into the cannula via the first opening. The cannula and the body member are connected in a non-linear angle. Also described is a method of using the minimally invasive high viscosity material delivery system.

### BRIEF DESCRIPTION OF THE DRAWINGS

FIG. 1 is a perspective, exploded view of one variation of the delivery system. This variation of the device may be directly filled with a high viscosity material.

FIG. 2 is a perspective, exploded view of another variation of the delivery system. This variation accepts high viscosity material from a removable, interchangeable container.

FIG. 3 is a perspective view of the described delivery system having an ergonomic grip.

### DETAILED DESCRIPTION

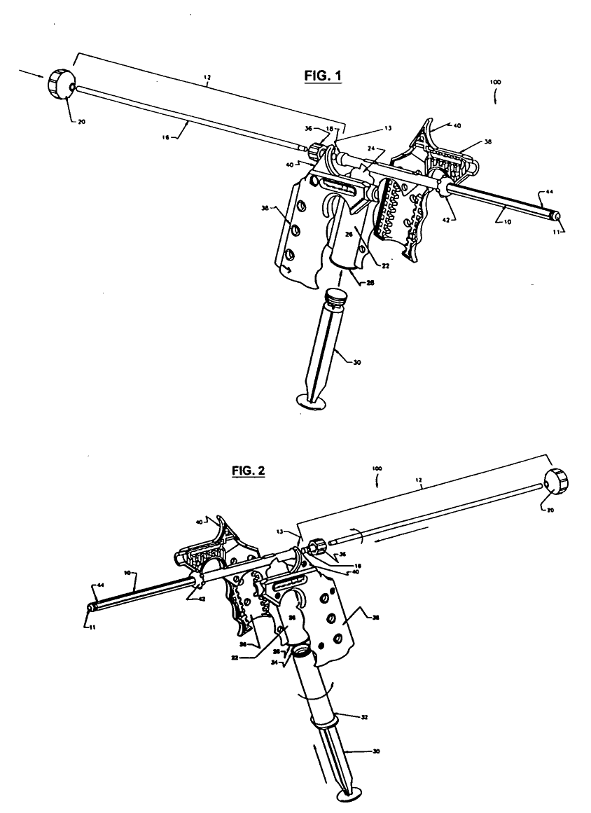

FIGS. 1-2 show a minimally invasive delivery system 100 for placement of a high viscosity material in the human body. The Figures show a system comprising a cannula 10 that dispenses a high viscosity material 14 (not shown) from an open delivery end or orifice 11 to the selected treatment site in the human body. The end of the cannula 10 opposite the delivery end 11 is associated with a dispenser 12. The dispenser 12 is placed so that it pressures the high viscosity material through and out of the cannula 10. The high viscosity material 14 may be a gel, putty, paste, flowable composition containing particulates, high viscosity liquid (e.g., more viscous than water or the like), a combination thereof, or the like. The cannula 10 may be constructed of a suitable material such as metal, metallic alloys, plastics, glass or the like capable of providing the strength needed to safely introduce the high viscosity material into the treatment site. We have found that stainless steel, polycarbonate, polypropylene, polyethylene, PTFE (Teflon) are quite suitable. The length and width and wall thickness of the cannula 10 may be varied depending, in general, upon the desired application. For instance, the length, inner diameter, and outer diameter of the cannula may be chosen to be, respectively, in the range of 5-35 cm, 1-20 mm, and 2-25 mm; or 7-30 cm, 1-15 mm, and 2-20 mm respectively; or 10-25 cm, 1-6 mm, and 2-10 mm.

The dispenser 12 is to provide pressure to the high viscosity material 14 in the cannula causing axial movement of the material through the cannula and metered delivery of the material through its delivery end 11. The dispensing pressure may be applied in a variety of ways, e.g., compressed gas (e.g., air, nitrogen or the like); manually, pneumatically, or hydraulically actuated plungers; or the like that is adapted for axial displacement of the high viscosity material 14 within the cannula 10. Referring again to FIG. 1, the dispenser 12 comprises a rod 16 having an optional tip 18 on one end and a knob or cap 20 on the other end. The rod 16, the tip 18, and the cap 20 may be constructed of a suitable material such as metal, metallic alloys, plastics, silicone, or the like. The rod 16 and the cap 20 may be constructed of stainless steel, polycarbonate, aluminum, PVC, ABS, acrylic, or the like and the tip 18 may be constructed of a rubbery Silicone such as sold by Merit Medical Systems, Inc. located in South Jordan, Utah.

Rod 16, in this variation, acts like a piston and slides through the interior passageway in cannula 10 displacing the viscous material found there through the distal delivery tip 11 of the cannula 10. The optional tip 18 on the end of rod 16 is simply a wiper and pressure seal and, as such, wipes the interior cannula wall of viscous material and maintains the pressure on the viscous material forward of the rod 16. Note that the cross sectional area of the rod 16 is fairly small to allow significant pressure to be generated at the tip 18 of the rod 16 upon imposition of a much more modest pressure upon knob 20.

Referring again to FIGS. 1-2, the delivery system 100 also comprises a body member 22 having a first opening 24 that is in communication with the cannula 10, a reservoir 26 for receiving the high viscosity material 14 and a second opening 28 through which the high viscosity material is placed in the reservoir. The second opening is associated with one or more transfer components (such as the plunger 30 shown in FIG. 1 and the plunger 30 and the associated container 32 shown in FIG. 2) used to move or to transfer the high viscosity material 14 from the body member 22 into the cannula 10 via the first opening 24. The body member 22 and the cannula 10 are connected in a non-linear angle. The body member 22 be constructed of a suitable material such as metal, metallic alloys, plastics, glass, or the like capable of withstanding the substantial pressures generated during use. The body member 22 may be constructed of stainless steel, polycarbonate, polypropylene, polyethylene, PTFE (Telfon), copolymer, or the like.

The transfer components 30 be of a variety of designs employing pressure sources such as compressed gas (e.g., air, nitrogen, or the like) or generated with a manually, pneumatically or hydraulically actuated plunger; or the like. The transfer components utilize the pressure to displace the high viscosity material 14 from the body member 22 through the first opening 24 into the cannula 10. For example, the transfer component 30 shown in FIG. 1 comprises a manually actuated plunger that moves axially through the second opening 28 displacing any high viscosity material through the reservoir 26 of the body member 22 thereby causing transfer of that high viscosity material 14 from the reservoir 26 into the cannula 10 via the first opening 24.

The rod 16, in most variations of the system, seals the first opening 24 as it presses the high viscosity material 14 from the cannula 10. Withdrawal of the tip of rod 16 is often needed to allow recharging the cannula 10 with additional high viscosity material 14 from the reservoir 26.

Referring to FIG. 1, the reservoir 26 may directly receive (i.e., be filled with) the high viscosity material 14. Alternatively, FIG. 2 shows a reservoir 26 that receives and is removably attachable to an interchangeable, perhaps disposable, container 32, filled with the high viscosity material 14. The container 32 may be attached via threads, luer lock or the like. The container 32 may be of a form such as a cartridge, ampoule, capsule, a syringe or the like. For example and referring to FIG. 2, the reservoir 26 is adapted to removably receive an open bore syringe (the interchangeable container 32) filled with the high viscosity material 14 via threads 34. Furthermore, leak prevention components or features such for leakage prevention of the high viscosity material 14 from the container 32 or the reservoir 26 may be optionally provided.

FIGS. 1-2 show the described device to have an angle between the axis of the cannula 10 and axis of the body member 22 to be non-linear. The value of the non-linear angle is chosen (e.g., from 1° to 179° or from 181° to 359°) based upon the desired applications. The non-linear angle may be in the range of 30° to 150° or from 210° to 330°; or from 50° to 130° or from 230° to 310°; or from 70° to 110° or 250° to 290°.

Referring to FIGS. 1-2, a seal 36 may be placed on the opening of body member 22 into which the rod 16 is inserted. Such a seal 36 may be removably attached tot he body member 22 and serves to help prevent the high viscosity material located in the cannula 10 from leaking out the cannula's non-dispensing end 13. Optionally, the seal 36 may take the form of a break-away hub. In this variation, the hub or seal 36 may be formed to cooperate with the rod 16 and the tip 18 in such a way that the tip 18 is sheared loose from the rod 16 as the rod 16 slides into the cannula 10 or upon some other designed user activity such as having the rod 16 and the break-away hub 36 connected via threads and applying a desired amount of torque to the rod 16, or the like. When the tip has been sheared from the rod 16, the rod 16 takes only one more trip to the end of the cannula 10 delivering viscous material. The sheared tip then remains in the delivery end 11 of the cannula 10 and prevents re-use and consequent reuse related contamination.

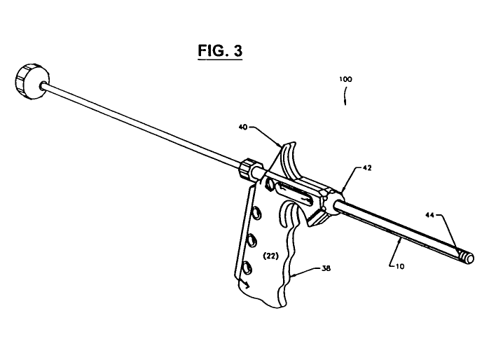

FIG. 3 provides a perspective view of the delivery system 100 and optional designs for physical handles. An ergonomic grip 38, syringe grip 40, and a split resistance cap 42 are shown. The cannula 10 and the body member 22 also may optionally include markers 44, preferably radiopaque markers, to provide for better visual inspection of the delivery process. The ergonomic grip 38 can be a design that is assembled from two sides, or a pair of shells as shown, affixed together with screws or the like or may be a single piece.

To prevent cross-contamination and need for clean up the entire delivery system 100 may be constructed of inexpensive, disposable materials and be disposed of when the reservoir is depleted. Alternatively, the delivery system 100 of the present invention may be cleaned and reused. If reuse is desired, it is preferred that the delivery device is constructed of materials that are autoclavable. Regardless of whether the delivery system 100 is disposable or autoclavable, it is preferred that each part of the delivery system 100 coming into contact with the high viscosity material 14 be chemically inert to the high viscosity material 14.

The accompanying figures and this description depict variations of the described minimally invasive high viscosity material delivery system and its components. Conventional fasteners such as snap fits, rivets, machine screws, nut and bolt connectors, machine threaded connectors, snap rings, clamps, toggles, pins, and the like may be used to connect the various components. Friction fitting, welding, or deformation, if suitable may be used as appropriate to connect the various components. Furthermore, materials for making the components of the system, unless otherwise specified, may be selected from appropriate materials such as metals, metallic alloys, fibers, plastics, and the like. Appropriate production methods may include casting, extruding, molding, machining, or the like.

The described system may be used to conduct a method a method for delivering high viscosity material comprising: providing the high viscosity material delivery system 100 described above; placing the high viscosity material 14 into the reservoir 26; transferring the high viscosity material 14 from the reservoir 26 into the cannula 10 via the first opening 24; and dispensing the high viscosity material 14 from the cannula 10 by introducing pressure to the cannula 10 from dispenser 12.

1. A delivery system for placing high viscosity material to a selected site in the human body, comprising:
a cannula having an axis and an opening for introducing high viscosity material to the selected site;
a dispenser for dispensing the high viscosity material from the cannula by introducing pressure upon the high viscosity material within the cannula;
a body member having a delivery axis and comprising a reservoir in fluid communication with the cannula, the body member being configured to transfer the high viscosity material from the reservoir into the cannula, and a second opening for receiving the high viscosity material into the reservoir; and
a transfer member in communication with the second opening, the transfer member configured for transferring the high viscosity material from the reservoir into the cannula via the first opening,
and wherein the cannula axis and the body member axis cross at a non-linear angle.

2. The system of claim 1 wherein the dispenser comprises a manually, pneumatically, or hydraulically actuated plunger.

3. The system of claim 1 wherein the dispenser comprises a rod axially slidable through the cannula.

4. The system of claim 1 wherein the dispenser comprises a rod attached to a tip on one end and a cap on the other end.

5. The system of claim 4 wherein the dispenser further comprises a break-away hub that is placed between the rod and the tip.

6. The system of claim 1 wherein the dispenser provides pressure by compressed gas.

7. The system of claim 1 wherein the transfer member comprises a manually, pneumatically or hydraulically actuated plunger.

8. The system of claim 1 wherein the transfer member provides pressure by compressed gas.

9. The system of claim 1 wherein the cannula has an inner diameter in the range of 1 mm to 20 mm.

10. The system of claim 1 wherein the cannula has an inner diameter in the range of 1 mm to 6 mm.

11. The system of claim 1 wherein the cannula has an outer diameter in the range of 2 mm to 10 mm.

12. The system of claim 1 wherein the cannula has a length of in the range of 5 cm to 35 cm.

13. The system of claim 1 wherein the cannula has a length in the range of 10 cm to 25 cm.

14. The system of claim 1 wherein the non-linear angle is in the range of 30 degrees to 150 degrees or 210 degrees to 330 degrees.

15. The system of claim 1 wherein the non-linear angle is in the range of 50 degrees to 130 degrees or 230 degrees to 310 degrees.

16. The system of claim 1 wherein the non-linear angle is in the range of 70 degrees to 110 degrees or 250 degrees to 290 degrees.

17. The system of claim 1 wherein the reservoir is adapted to removably receive a container filled with a high viscosity material.

18. The system of claim 17 wherein the container is removably received by the reservoir by means of threads or luer lock.

19. The system of claim 18 wherein the container is a syringe, cartridge, ampoule, or capsule.

20. The system of claim 1 wherein the reservoir is directly fillable with a high viscosity material.

21. The system of claim 1 further comprising physical handles.

22. The system of claim 21 wherein the physical handles comprise an ergonomic grip, a syringe grip, or a combination thereof.

23. The system of claim 1 further comprising a split resistance cap.

24. The system of claim 1 wherein the cannula further comprises markers.

25. The system of claim 24 wherein the markers are radiopaque.

26. The system of claim 1 wherein the body member further comprises markers.

27. The system of claim 26 wherein the markers are radiopaque.

28. The system of claim 1 wherein the cannula and the body member comprise stainless steel, polycarbonate, polypropylene, polyethylene, PTFE, or a combination thereof.

29. A minimally invasive tissue regeneration material delivery system comprising:
a cannula;
a dispenser for dispensing the tissue regeneration material comprising a cap, rod, a breakaway hub, and a tip;
a body member having a first opening that is in communication with the cannula, a reservoir for removably receiving the tissue regeneration material, and a second opening; and
an open bore syringe filled with the tissue regeneration material that is removably connected to the reservoir;
a plunger for transferring the tissue regeneration material from the body member into the cannula via the first opening,
wherein the dispenser is associated with the cannula, the plunger is in communication with the second opening, and the cannula, and the body member are connected in a non-linear angle in the range of 70 degrees to 110 degrees or 250 degrees to 290 degrees.

30. A method for delivering high viscosity material comprising:
1.) providing a high viscosity material delivery system comprising:
a.) a cannula;
b.) a dispenser for dispensing a high viscosity material;
c.) a body member having a first opening that is in communication with the cannula, a reservoir for receiving a high viscosity material, and a second opening; and
d.) a transfer member for transferring the high viscosity material from the body member into the cannula via the first opening, wherein the dispenser is associated with the cannula, the transfer member is in communication with the second opening and the cannula and the body member are connected in a non-linear angle;
2.) placing the high viscosity material into the reservoir;
3.) actuating the transfer member to transfer the high viscosity material from the reservoir into the cannula via the first opening; and
4.) actuating the dispenser to dispense the high viscosity material from the cannula.
